SEQUENCE LISTING (6 sequences)

SEQ ID NO: 1 - DNA, 615 bp, Saccharomyces cerevisiae
  misc_feature: LOC1 YFR001W SGDIDS000001897, chrVI149110..149724
atggcaccaaagaaaccttctaagagacaaaatctgagaagagaagtcgcaccagaggtgtttcaagattcacaagctaggaatcaactagcgaatgttcctcatcttaccgaaaaatctgcccagcgtaagccttctaaaaccaaggttaaaaaagaacagtctttggctagactttatggtgcgaagaaggacaagaaggggaaatattctgagaaagacttgaatattccaacactcaatagagctatcgttccgggtgttaaaataaggaggggaaagaaaggtaagaaattcattgctgataacgacactctgactttaaaccgtttaataacaactattggtgacaagtacgacgatatagctgagagtaagcttgaaaaggctagaagattagaagagatacgagaattgaaaagaaaggaaattgaaagaaaggaagcgcttaaacaagataaactagaagaaaaaaaagacgagattaaaaagaagtcttctgtcgcaaggactatacgtagaaagaataaacgtgatatgttgaaaagtgaagcaaaagctagtgaaagtaaaactgaaggaaggaaggtaaaaaaagtctcatttgctcaatag

SEQ ID NO: 2 - protein, 204 aa, Saccharomyces cerevisiae [sequence omitted]

SEQ ID NO: 3 - DNA, 591 bp, Saccharomyces cerevisiae
  misc_feature: SMB1 YER029C SGDIDS000000831, chrV212587..213177
atgagcaaaatacaggtggcacatagcagccgactagccaaccttattgattataagctgagggttctcactcaagatggccgcgtttacatcgggcaattgatggcatttgataaacatatgaatttagtgttgaatgagtgtatagaagagagggtacccaaaactcaactagataaattaagaccgagaaaagattcaaaagatggaaccactttgaacatcaaggtagaaaaaagagtgttgggactgactatactaagaggagaacagatcttatccacagtggtggaggataagccgctactatccaagaaggaaagactagtgagagataaaaaggaaaagaaacaagcgcaaaagcagacgaaactaagaaaagagaaagagaaaaagccgggaaagatcgctaaacctaacacggccaatgcgaagcatactagtagcaattctagggagattgcccaaccatcgtcgagcagatacaatggtggcaacgataatatcggcgcaaataggtcgaggtttaataatgaagcgccccctcaaacaaggaagtttcagcccccaccaggttttaaaagaaaataa

SEQ ID NO: 4 - protein, 196 aa, Saccharomyces cerevisiae
  MISC_FEATURE: SMB1; UniProtKB - P40018
MSKIQVAHSSRLANLIDYKLRVLTQDGRVYIGQLMAFDKHMNLVLNECIEERVPKTQLDKLRPRKDSKDGTTLNIKVEKRVLGLTILRGEQILSTVVEDKPLLSKKERLVRDKKEKKQAQKQTKLRKEKEKKPGKIAKPNTANAKHTSSNSREIAQPSSSRYNGGNDNIGANRSRFNNEAPPQTRKFQPPPGFKRK

SEQ ID NO: 5 - DNA, 801 bp, Saccharomyces cerevisiae
  misc_feature: BUD13 YGL174W SGDIDS000003142, chrVII174545..175345
atggcattgcatcagtatttatcagagacttatgggcccacgaaacccaaaaataagacgaaaaagaagaagaaagagtcaaaatcagacgctaactcagacaaaacttctttgatagtaaaagaacggctaagtacactgcaacaagaacaggagaagtcaggagttgcttcattcagcaagtttgacaaacaaaaaagcaagaatatatggaagaacctggaaacaaacgagctttcccatgcaataacacatccttccgcatcgtcaattactggcaacgaaagcaagaacgatctaaaggaaatcagggctcaagagccacttgtcacagtagcagacaaatcgaaaacacgaaaaaccatatacagagacgctcaaggtcacaagattcaggaagattccaagatagacgattctagttttagtcgatctaaatatgaagatgagaaagccgcggaaagagagcaatacctgaaaaatttgaatatgggagacgtgcaaaagcttggaataaatgtagatgcacatgataagaagaaaaatcaaactgcctcgagtctgacgatagaagaccctgcaataacatttacacatgacaaagaaagaactgtaaaaacatctttactgggccgcaagctttatgataagccagcacctgagaacaggtttgccattatgcctgggtcaagatgggacggtgtccacagatcaaatggctttgaagaaaaatggtttgctaagcaaaatgagatcaatgagaagaaagtgcaaagctacaccctacaggaggattattga

SEQ ID NO: 6 - protein, 266 aa, Saccharomyces cerevisiae
  MISC_FEATURE: BUD13; UniProtKB - P46947
MALHQYLSETYGPTKPKNKTKKKKKESKSDANSDKTSLIVKERLSTLQQEQEKSGVASFSKFDKQKSKNIWKNLETNELSHAITHPSASSITGNESKNDLKEIRAQEPLVTVADKSKTRKTIYRDAQGHKIQEDSKIDDSSFSRSKYEDEKAAEREQYLKNLNMGDVQKLGINVDAHDKKKNQTASSLTIEDPAITFTHDKERTVKTSLLGRKLYDKPAPENRFAIMPGSRWDGVHRSNGFEEKWFAKQNEINEKKVQSYTLQEDY

(19) United States
(12) Patent Application Publication
(10) Pub. No.: US 2022/0259604 A1
(43) Pub. Date: Aug. 18, 2022

(54) MODIFIED YEAST CELLS THAT OVER-EXPRESS SELECTED ENDOGENOUS PROTEINS

(71) Applicant: DANISCO US INC, Palo Alto, CA (US)

(72) Inventors: Jaclyn Diana DEMARTINI, Palo Alto, CA (US); Celia Emily Gaby PAYEN, WILMINGTON, DE (US)

(21) Appl. No.: 17/623,086

(22) PCT Filed: Jun. 22, 2020
(86) PCT No.: PCT/US2020/038884

§ 371 Date: Dec. 27, 2021

Related U.S. Application Data
(60) Provisional application No. 62/868,295, filed on Jun. 28, 2019.

(51) Int. Cl. C12N 15/52; A23K 10/16; A23K 20/147; C12P 13/08; C07K 14/39; C12P 7/06

(52) U.S. Cl. CPC C12N 15/52; A23K 10/16; C12P 7/06; C12P 13/08; C07K 14/39; A23K 20/147

(57) ABSTRACT

The present strains and methods relate to yeast cells that over-overproduce selected endogenous proteins having a high amino acid content of a selected amino acid. The yeast can be used in a conventional bioethanol production facility to produce alcohol along with increased amounts of a selected amino acid, resulting in increased quality and commercial value of fermentation products and co-products, such as animal feed ingredients.

### TECHNICAL FIELD

The present strains and methods relate to yeast cells that over-overproduce selected endogenous proteins having a high amino acid content of a selected amino acid. The yeast can be used in a conventional bioethanol production facility to produce alcohol along with increased amounts of a selected amino acid, resulting in increased quality and commercial value of fermentation products and co-products, such as animal feed ingredients.

### BACKGROUND

Many countries make fuel alcohol from fermentable substrates, such as corn starch, sugar cane, cassava, and molasses. According to the Renewable Fuel Association (Washington D.C., United States), 2015 fuel ethanol production was close to 15 billion gallons in the United States, alone.

In addition to producing about 2.8 gallons of ethanol, a 56-pound bushel of corn processed in a dry mill ethanol plant also generates about 17.5 pounds of animal feed. Animal feed is usually in the form of distillers dried grains with solute (DDGS) and represents the starch-depleted portion of corn plus the biomass of the yeast used for fermentation. Per weight, DDGS is more nutritional for animals than the unprocessed corn because it is richer in protein and fat. Beyond DDGS, dry mill ethanol plants also have the ability to create other protein-rich corn products and co-products for animal feed applications.

Traditionally, lysine, histidine, isoleucine, leucine, valine, methionine, phenylalanine, threonine, and tryptophan have been classified as essential amino acids for non-ruminant animals. Cysteine and tyrosine can be synthesized from methionine and phenylalanine respectively, but both precursors are essential amino acids. If these amino acids cannot be supplied in adequate amounts in DDGS to meet feed conversion expectations, they must be supplemented. Synthetic lysine, in particular, can represent a significant cost of animal feed.

The need exists for ways to improve or maintain the production of alcohol from starch-containing feedstocks while increasing the nutritional value of animal feed co-products.

### SUMMARY

Described are compositions and methods relating to yeast cells that over-overproduce selected endogenous proteins having a high amino acid content of a selected amino acid. The yeast can be used in a conventional bioethanol production facility to produce alcohol along with increased amounts of a selected amino acid, resulting in increased quality and commercial value of fermentation products and co-products, such as animal feed ingredients. Aspects and embodiments of the compositions and methods are described in the following, independently-numbered paragraphs.

1. In one aspect, a microorganism for use in preparing a food or feed composition is provided, comprising a genetic modification that increases the expression of an endogenous gene encoding a protein having an elevated ratio of a preselected amino acid relative to the total amino acid content of the protein, wherein the preselected amino acid confers a nutritional benefit to the food or feed composition compared an otherwise identical food or feed composition comprising an otherwise identical microorganism, or product derived therefrom, lacking the genetic modification.

2. In some embodiments of the microorganism of paragraph 1, the endogenous gene is naturally present in the microorganism prior to introducing the genetic modification.

3. In some embodiments of the microorganism of paragraph 1 or 2, the genetic modification is the introduction of an expression cassette comprising an additional copy of the endogenous gene.

4. In some embodiments of the microorganism of paragraph 1 or 2, the genetic modification is the introduction of a stronger promoter operably-linked to the endogenous gene.

5. In some embodiments of the microorganism of paragraph 1 or 2, the genetic modification is the deletion of a naturally-present negative regulator of expression of the endogenous gene, or wherein the genetic modification increases the expression of a naturally-present positive regulator of expression of the endogenous gene.

6. In some embodiments of the microorganism of any of paragraphs 1-5, the elevated ratio of the preselected amino acid relative to the total amino acid content of the protein is at least 1.2 compared to the ratio of the preselected amino acid relative to the total amino acid content of all proteins produced by the microorganism.

7. In some embodiments of the microorganism of any of paragraphs 1-6, the organism is an ethanolagen.

8. In some embodiments of the microorganism of any of paragraphs 1,7, the organism is a Saccharomyces sp.

9. In some embodiments of the microorganism of any of paragraphs 1-8, the microorganism does not comprise an exogenous gene encoding a protein having an elevated of a preselected amino acid relative to the total amino acid content of the protein that is introduced for the purpose of, conferring a nutritional benefit to the food or feed composition.

10. In some embodiments of the microorganism of any of paragraphs 1-9, the cells further comprise an exogenous gene encoding a carbohydrate processing enzyme, one or more genes of the phosphoketolase pathway, an alteration in the glycerol pathway and/or the acetyl-CoA pathway, or an alternative pathway for making ethanol.

11. In another aspect, a method for increasing the nutritional value of a microorganism, or product derived therefrom, in a food or feed composition is provided, comprising introducing into the microorganism a genetic modification that increases the expression of an endogenous gene encoding a protein having an elevated ratio of a preselected amino acid relative to the total amino acid content of the protein, wherein the preselected amino acid confers a nutritional benefit to the food or feed composition compared an otherwise identical food or feed composition comprising an otherwise identical microorganism, or product derived therefrom, lacking the genetic modification.

12. In some embodiments of the method of paragraph 11, the endogenous gene is naturally present in the microorganism prior to introducing the genetic modification.

13. In some embodiments of the method of paragraph 11 or 12, the genetic modification is the introduction of an expression cassette comprising an additional copy of the endogenous gene.

14. In some embodiments of the method of paragraph 11 or 12, the genetic modification is the introduction of a stronger promoter operably-linked to the endogenous gene.

15. In some embodiments of the method of paragraph 11 or 12, the genetic modification is the deletion of a naturally-present negative regulator of expression of the endogenous gene, or wherein the genetic modification increases the expression of a naturally-present positive regulator of expression of the endogenous gene.

16. In some embodiments of the method of any of paragraphs 11-15, the elevated ratio of the preselected amino acid relative to the total amino acid content of the protein is at least 1.2 compared to the ratio of the preselected amino acid relative to the total amino acid content of all proteins produced by the microorganism.

17. In some embodiments of the method of any of paragraphs 11-16, the microorganism is an ethanolagen.

18. In some embodiments of the method of any of paragraphs 11-17, the organism is a Saccharomyces sp.

19. In some embodiments of the method of any of paragraphs 11-18, the microorganism does not comprise an exogenous gene encoding a protein having an elevated ratio of a preselected amino acid relative to the total amino acid content of the protein that is introduced for the purpose of conferring a nutritional benefit to the food or feed composition.

20. In some embodiments of the method of any of paragraphs 11-19, the cells further comprise an exogenous gene encoding a carbohydrate processing enzyme, one or more genes of the phosphoketolase pathway, an alteration in the glycerol pathway and/or the acetyl-CoA pathway, or an alternative pathway for making ethanol.

These and other aspects and embodiments of present modified cells and methods will be apparent from the description, including any accompanying Figures.

### DETAILED DESCRIPTION

### I. Overview

Described are methods relating to yeast having a genetic mutation that relate to yeast cells that over-overproduce selected endogenous proteins having a high amino acid content of a selected amino acid. The yeast can be used in a conventional bioethanol production facility to produce alcohol along with increased amounts of a selected amino acid, resulting in increased quality and commercial value of fermentation products and co-products, such as animal feed ingredients.

### Definitions

Prior to describing the present strains and methods in detail, the following terms are defined for clarity. Terms not defined should be accorded their ordinary meanings as used in the relevant art.

As used herein, “alcohol” refers to an organic compound in which a hydroxyl functional group (—OH) is bound to a saturated carbon atom.

As used herein, “yeast cells” yeast strains, or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases (see above), refer to yeast that include genetic modifications and characteristics described herein. Variant/modified yeast do not include naturally occurring yeast.

As used herein, the phrase “substantially free of an activity,” or similar phrases, means that a specified activity is either undetectable in an admixture or present in an amount that would not interfere with the intended purpose of the admixture.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, an “endogenous” gene or protein originates from within a system in question, such as a yeast cell. Such a gene or protein is present naturally and without human intervention. As used herein, even though an endogenous gene or protein may be over-expressed, it is still considered to be endogenous if some amount of the gene or protein is naturally present.

As used herein, an “exogenous” gene or protein originates from outside a system in question, such as a yeast cell. Such a gene or protein is not present naturally and must be introduced, e.g., through human intervention. As used herein, even though an expression cassette may be introduced to over-produce an endogenous gene or protein, the gene or protein is not considered to be exogenous if some amount is naturally present.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444: programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson at al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF. |

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the terms “wild-type” and “native” are used interchangeably and refer to genes proteins or strains found in nature.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, the term “expressing a polypeptide” and similar terms refers to the cellular process of producing a polypeptide using the translation machinery (e.g., ribosomes) of the cell.

As used herein, “over-expressing a polypeptide,” “over-producing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes a promoter, and amino acid coding region and a terminator (i.e., promoter::amino acid coding region::terminator) and other nucleic acid sequence needed to allow the encoded polypeptide to be produced in a cell. Expression cassettes can be exogenous (i.e., introduced into a cell) or endogenous (i.e., extant in a cell).

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements.

As used herein, “disruption of a gene” refers broadly to any genetic or chemical manipulation, i.e. mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using RNAi, antisense, or any other method that abolishes gene expression. A gene can be disrupted by deletion or genetic manipulation of non-adjacent control elements.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “primarily genetic determinant” refers to a gene, or genetic manipulation thereof, that is necessary and sufficient to confer a specified phenotype in the absence of other genes, or genetic manipulations, thereof. However, that a particular gene is necessary and sufficient to confer a specified phenotype does not exclude the possibility that additional effects to the phenotype can be achieved by further genetic manipulations.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “fermentation broth” is the product of an ethanol production facility following fermentation with yeast but prior to distillation.

As used herein, “whole stillage” is the byproduct an ethanol production facility following distillation.

As used herein, “thin stillage” is the liquid portion of whole stillage following separation of solid materials.

As used herein, “distillers' grains (DG)” is the solid/slurry component of whole stillage.

As used herein, “distillers' dried grains (DDG) is DG that have been dried.

As used herein, “distillers' dried grains with solutes (DDGS) is DG that has been dried along with the concentrated thin stillage for added nutritional value.

As used herein, a “wet” by-product of distillation contains at least 20% water by weight.

As used herein, a “dried” by-product of distillation contains less than 20% water by weight.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:

| | |
|---|---|
| ° C. | degrees Centigrade |
| DG | distillers' grains |
| DDG | distillers' dried grains |
| DDGS | distillers' dried grains with solutes |
| DNA | deoxyribonucleic acid |
| DP | degree of polymerization |
| DS | dry solids |
| EtOH | ethanol |
| g or gm | gram |
| g/L | grams per liter |
| GA | glucoamylase |
| GAU/g DS | glucoamylase units per gram dry solids |
| HPLC | high performance liquid chromatography |
| hr or h | hour |
| kDa | kilodalton |
| M | molar |
| mg | milligram |
| mL or ml | milliliter |
| ml/min | milliliter per minute |
| mM | millimolar |
| N | normal |
| na | not applicable |
| PCR | polymerase chain reaction |
| ppm | parts per million |
| SAPU/g DS | protease units per gram dry solids |
| SSCU/g DS | fungal α-amylase units per gram dry solids |
| Δ | relating to a deletion |
| μg | microgram |
| μL and μl | microliter |
| μM and μm | micromolar |

### III. Yeast Cells Expressing Increased Amounts of Pre-Selected Endogenous Proteins

U.S. Pat. No. 7,309,602 describes a method for increasing the value of fermentation residuals by introducing into yeast cells a recombinant expression vector encoding a polypeptide comprising essential amino acids. While a plausible strategy for producing fermentation products or co-products that contain increased amounts of valuable amino acids, it often requires extensive work to identify valuable proteins that are well-expressed in, and well-tolerated by, yeast.

The present compositions and methods represent an improved strategy toward the production of valuable proteins. Rather than select an exogenous protein of interest that contains a high ratio of amino acids of interest, knowledge about the amino acid content of endogenous yeast protein is used to select protein that can be over-expressed to produce similar results.

The amino acid content of every protein produced by an organism such a Saccharomyces cerevisiae can determined using readily-available information. As an example, the average occurrence of lysine as a fraction of total residues in all S. cerevisiae protein was found to be 0.08 (or 8%), which is notably greater than the 5% expected if all amino acid residues were equally represented. Five lysine-rich proteins which were identified in the present study are shown in Table 1. These proteins were lysine-rich and, based on their annotations (see, below), seemed unlikely to be toxic to the cell if overexpressed. The gene encoding the protein, total length of the protein, number of lysine residues and fraction lysine (expressed as K/AA) are indicated.

TABLE 1
Selected lysine rich proteins in S. cerevisiae

| Protein | Gene | K/AA |
|---|---|---|
| LOC1 | YFR001W | 0.200 |
| MRPL24 | YMR193W | 0.151 |
| BUD13 | YGL174W | 0.146 |
| SYF2 | YGR129W | 0.144 |
| SMB1 | YER029C | 0.142 |

Similar analysis can be performed for any preselected amino acid, most importantly amino acids that are essential to animals. For the purposes of the present study, amino acid composition data from 5,895 S. cerevisiae proteins was compiled, allowing the identification of proteins rich in any one or more selected amino acids.

In some embodiments, the elevated ratio of the preselected amino acid in the endogenous protein is at least 1.2, at least 1.4, at least 1.6, at least 1.8, or even at least 2.0 in terms of the selected amino acid as a fraction of total amino acids compared to the fraction of the amino acid in total cellular protein. In some embodiments, the amount of the preselected amino acid in the endogenous protein is at least 20%, at least 40%, at least 60%, at least 80%, or even at least 100% greater in terms of the amount of the amino acid present in total cellular protein.

In some embodiments, the increase expression of the endogenous, selected-amino-acid-rich proteins produced by the modified cells is at least 0.5-fold, at least 1.0-fold, at least 1.5-fold, at least 2.0-fold, at least 3.0-fold, or more, compared to the amount of endogenous, selected-amino-acid-rich proteins produced by parental cells grown under the same conditions.

Preferably, increased endogenous, selected-amino-acid-rich protein-expression is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the present compositions and methods involve introducing into yeast cells a nucleic acid capable of directing the over-expression, or increased expression, of an endogenous, selected-amino-acid-rich protein. Particular methods include but are not limited to (i) introducing an exogenous expression cassette for producing the polypeptide into a host cell, optionally in addition to an endogenous expression cassette, (ii) substituting an exogenous expression cassette with an endogenous cassette that allows the production of an increased amount of the polypeptide, (iii) modifying the promoter of an endogenous expression cassette to increase expression, (iv) increase copy number of the same or different cassettes for over-expression of endogenous lysine-rich polypeptides, and/or (v) modifying any aspect of the host cell to increase the half-life of the polypeptide in the host cell.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

In some embodiments, the parental cell that is modified already includes an engineered pathway of interest, such as a PKL pathway to increases ethanol production, or any other pathway to increase alcohol production.

Where the preselected amino acid is lysine, as exemplified, potential endogenous proteins include LOC1, a 60S ribosomal subunit assembly/export protein, SMB1, a small nuclear ribonucleoprotein-associated protein, BUD13, a pre-mRNA-splicing factor MRPL24, a mitochondrial ribosomal protein and SYF2, a pre-mRNA splicing factor.

The amino acid sequence of the exemplified LOC1 polypeptide is shown, below, as SEQ ID NO: 2:

MAPKKPSKRQ NLRREVAPEV FQDSQARNQL ANVPHLTEKS
AQRKPSKTKV KKEQSLARLY GAKKDKKGKY SEKDLNIPTL
NRAIVPGVKI RRGKKGKKFI ADNDTLTLNR LITTIGDKYD
DIAESKLEKA RRLEEIRELK RKEIERKEAL KQDKLEEKKD
EIKKKSSVAR TIRRKNKRDM LKSEAKASES KTEGRKVKKV
SFAQ

The amino acid sequence of the exemplified SMB1 polypeptide is shown, below, as SEQ ID NO: 4:

MSKIQVAHSS RLANLIDYKL RVLTQDGRVY IGQLMAFDKH
MNLVLNECIE ERVPKTQLDK LRPRKDSKDG TTLNIKVEKR
VLGLTILRGE QILSTVVEDK PLLSKKERLV RDKKEKKQAQ
KQTKLRKEKE KKPGKIAKPN TANAKHTSSN SREIAQPSSS
RYNGGNDNIG ANRSRFNNEA PPQTRKFQPP PGFKRK

The amino acid sequence of the exemplified BUD13 polypeptide is shown, below, as SEQ ID NO: 6:

MALHQYLSET YGPTKPKNKT KKKKKESKSD ANSDKTSLIV
KERLSTLQQE QEKSGVASFS KFDKQKSKNI WKNLETNELS
HAITHPSASS ITGNESKNDL KEIRAQEPLV TVADKSKTRK
TIYRDAQGHK IQEDSKIDDS SFSRSKYEDE KAAEREQYLK
NLNMGDVQKL GINVDARDKK KNQTASSLTI EDPAITFTHD
KERTVKTSLL GRKLYDKPAP ENRFAIMPGS RWDGVHRSNG
FEEKWFAKQN EINEKKVQSY TLQEDY

In some embodiments of the present compositions and methods, the amino acid sequence of the LOC1, SMB1 or BUD13 polypeptide that is over-expressed in modified yeast cells has at least about 70%, at least about 75%, at least about 80%, at least about 85%, at least about 87%, at least about 90%, at least about 91%, at least about 92%, at least about 93%, at least about 94%, at least about 95%, at least about 96%, at least about 97%, at least about 98%, or even at least about 99% identity, to SEQ ID NO: 2, SEQ ID NO: 4 or SEQ ID NO: 6.

In some embodiments, the modified cells include other genes or other modifications that increase lysine production.

### IV. Combined Expression of Selected Amino-Acid-Rich Endogenous Proteins with Mutations that Benefit Alcohol Production

In some embodiments, in addition to producing increased amounts of endogenous, selected-amino-acid-rich proteins, the present modified yeast cells further include additional modifications that benefit alcohol production.

In particular embodiments the modified yeast cells include an artificial of alternative ethanol-producing pathway resulting from the introduction of a heterologous phosphoketolase (PKL) gene, a heterologous phosphotransacetylase (PTA) gene and a heterologous acetylating acetyl dehydrogenase (AADH), as described in WO2015148272 (Miasnikov et al.), to channel carbon flux away from the glycerol pathway and toward the synthesis of acetyl-CoA, which is then converted to ethanol.

The modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.).

In some embodiments, the present modified yeast cells may further overexpress a sugar transporter-like (STL1) polypeptide (see. e.g., Ferreira et al. (2005) Mol Biol Cell 16:2068-76; Dušková et al. (2015) Mol Microbiol 97:541-59 and WO 2015023989 A1) to increase ethanol production and reduce acetate.

In some embodiments, the present modified yeast cells may further overexpress a polyadenylate-binding protein, e.g., PAB1, to increase alcohol production and reduce acetate production.

In some embodiments, the present modified yeast cells further comprise a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1 GPD2, BDH1, and YMR226C.

### V. Combined Expression of Selected Amino-Acid-Rich Endogenous Proteins with Other Beneficial Mutations

In some embodiments, in addition to producing increased amounts of endogenous, selected-amino-acid-rich proteins, endogenous lysine-rich proteins, optionally in combination with genetic modifications that benefit alcohol production, the present modified yeast cells further include any number of additional genes of interest encoding proteins of interest. Additional genes of interest may be introduced before, during, or after genetic manipulations that result in reduced lysine feedback inhibition or increased alcohol production. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, isoamylase, a cellulase, a trehalase, a lipase, a pectinase, a polyesterase, a cutinase, an oxidase, a transrase, a reductase, a hemicellulase, a mannanase, an esterase, an isomerase, a pectinases, a lactase, a peroxidase and a laccase. Proteins of interest may be secreted, glycosylated, and otherwise-modified.

### VI. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Numerous yeast have been genetically engineered to produce heterologous enzymes or even to include heterologous pathways.

### VII. Substrates and Conditions

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Numerous variations of ethanol production process exist, including cold cook, or no cook, involving liquefaction at or below the gelatinization temperature, simultaneous saccharification and fermentation, fractionation processes, and the like. None are expected to be incompatible with the present compositions and methods.

### VIII. Fermentation Products and Co-Products

Typical alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

Valuable by-products (or co-products) of alcohol production, and particularly dry-grind ethanol production, are products for animal feed, usually in the form of distillers' dried grains (DDG) or, more commonly, distillers' dried grains with solutes (DDGS). Such animal feed products are in many ways more nutritional than the initial feed-stocks used for ethanol production as they are depleted for carbohydrates but enriched for amino acids derived both from the feed-stock and the fermenting organism (i.e., ethanolagen).

The specific amino acid composition of DDGS or other corn co-product is important to the quality of animal feed as some amino acids are far more important than others. Lysine is an essential amino acid for most farm animals and, if not provided in adequate amounts by adequately by DDG, DDGS, or other post fermentation co-products, must be supplemented to maximize feed conversion. Synthetic lysine is expensive and represents a significant cost of animal feed.

Because yeast represent a significant component of post-fermentation products, the amino acid content of the yeast significantly affects the amino acid content of fermentation broth, whole stillage, thin stillage, distillers dried grains, distillers dried grains with solutes, condensed distillers solubles or other protein-containing post fermentation coproducts. Replacing convention yeast with the present yeast increases the amounts of lysine in such post-fermentation products, thereby increasing their value as animal feed products.

Using the present modified yeast, an increase in lysine of at least 0.2-fold, at least 0.5-fold, at least 1.0-fold, at least 1.2-fold, at least 1.5-fold, at least 1.7-fold, at least 2.0-fold, or more, can be realized.

These and other aspects and embodiments of the present strains and methods will be apparent to the skilled person in view of the present description. The following examples are intended to further illustrate, but not limit, the strains and methods.

### EXAMPLES

### Example 1: Selection of Genes Encoding Endogenous Lysine-Rich Proteins

The amino acid content of every protein encoded by an endogenous gene in the S. cerevisiae genome was analyzed to identify native proteins for enriched for lysine. The average occurrence of lysine as a fraction of total residues (i.e., K/AA) in a protein was found to 0.08. Therefore, S. cerevisiae proteins typically had 8% lysine content, which is greater than the 5% expected if all amino acid residues were equally represented. The top five candidate genes for lysine overproduction are summarized in Table 2, below. These genes did not necessarily have the highest K/AA; however, based on public annotation, they seemed most likely to be tolerated by cells if overexpressed.

TABLE 2
Selected lysine rich proteins in Saccharomyces cerevisiae

| Gene | Protein | AA length | Number K | K/AA |
|---|---|---|---|---|
| YFR001W | LOC1 | 205 | 41 | 0.200 |
| YMR193W | MRPL24 | 259 | 39 | 0.151 |
| YGL174W | BUD13 | 267 | 39 | 0.146 |
| YGR129W | SYF2 | 216 | 31 | 0.144 |
| YER029C | SMB1 | 197 | 28 | 0.142 |

The expression of each gene in Table 2 was determined at various time during aerobic growth of FERMAX™ Gold (Martrex Inc., Minn., USA; herein abbreviated, “FG”), a well-known fermentation yeast used in the grain ethanol industry. RNA was prepared from individual samples according to the TRIzol method (Life-Tech, Rockville, Md.). The RNA was then cleaned up with Qiagen RNeasy Mini Kit (Qiagen, Germantown, Md.). The cDNA from total mRNA in individual samples was generated using Applied Biosystems High Capacity cDNA Reverse Transcription kit (Thermo Fisher Scientific, Wilmington, Del.). The prepared cDNA of each sample was sequenced using the shotgun method, and then quantified with respect to individual genes. The results are reported in Table 3 as reads per kilobase ten million transcripts (RPK10M), and can be used to quantify the amount of each transcript in a sample.

TABLE 3
Expression of lysine proteins in FG

| | RNASeq expression in FG | | | |
|---|---|---|---|---|
| Gene | 12 hr | 24 hr | 48 hr | 55 hr |
| YFR001W | 1232.612 | 219.905 | 152.232 | 117.731 |
| YMR193W | 1111.334 | 598.860 | 141.213 | 112.909 |
| YGL174W | 332.657 | 385.651 | 146.979 | 130.690 |
| YGR129W | 324.01 | 230.55 | 66.26 | 69.15 |
| YER029C | 1615.890 | 451.956 | 116.614 | 139.973 |

Based on several factors, including the lysine content of the proteins, expression levels, overall length of the gene and ease of amplification by PCR, three genes were selected for further study, namely, (i) YFR001W, encoding LOC1, a 60S ribosomal subunit assembly/export protein, (ii) YER029C, encoding SMB1, a small nuclear ribonucleoprotein-associated protein, and (iii) YGL174W, encoding BUD13, a pre-mRNA-splicing factor.

### Example 2: Over-Production Lysine-Rich Proteins in Yeast

Using standard molecular techniques, LOC1, BUD13 and SMB1 were over-expressed in FG using a strong promoter (FBA1) from expression cassettes inserted at a preselected locus. All procedures were based on the publicly available nucleic acid sequence of YFR001w, YER029c and YGL174w which are provided below (5′ to 3′):

>LOC1 YFR001W SGDID:S000001897,
chrVI:149110 . . . 149724 (SEQ ID NO: 1):
ATGGCACCAAAGAAACCTTCTAAGAGACAAAATCTGAGAAGAGAAGTCGC
ACCAGAGGTGTTTCAAGATTCACAAGCTAGGAATCAACTAGCGAATGTTC
CTCATCTTACCGAAAAATCTGCCCAGCGTAAGCCTTCTAAAACCAAGGTT
AAAAAAGAACAGTCTTTGGCTAGACTTTATGGTGCGAAGAAGGACAAGAA
GGGGAAATATTCTGAGAAAGACTTGAATATTCCAACACTCAATAGAGCTA
TCGTTCCGGGTGTTAAAATAAGGAGGGGAAAGAAAGGTAACAAATTCATT
GCTGATAACGACACTCTGACTTTAAACCGTTTAATAACAACTATTGGTGA
CAAGTACGACGATATAGCTGAGAGTAAGCTTGAAAAGGCTAGAAGATTAG
AAGAGATACGAGAATTGAAAAGAAAGGAAATTGAAAGAAAGGAAGCGCTT
AAACAAGATAAACTAGAAGAAAAAAAAGACGAGATTAAAAAGAAGTCTTC
TGTCGCAAGGACTATACGTAGAAAGAATAAACGTGATATGTTGAAAAGTG
AAGCAAAAGCTAGTGAAAGTAAAACTGAAGGAAGGAAGGTAAAAAAAGTC
TCATTTGCTCAATAG
>SMB1 YER029C SGDID:S000000831,
chrV:212587 . . . 213177 (SEQ ID NO: 3):
ATGAGCAAAATACAGGTGGCACATAGCAGCCGACTAGCCAACCTTATTGA
TTATAAGCTGAGGGTTCTCACTCAAGATGGCCGCGTTTACATCGGGCAAT
TGATGGCATTTGATAAACATATGAATTTAGTGTTGAATGAGTGTATAGAA
GAGAGGGTACCCAAAACTCAACTAGATAAATTAAGACCGAGAAAAGATTC
AAAAGATGGAACCACTTTGAACATCAAGGTAGAAAAAAGAGTGTTGGGAC
TGACTATACTAAGAGGAGAACAGATCTTATCCACAGTGGTGGAGGATAAG
CCGCTACTATCCAAGAAGGAAAGACTAGTGAGAGATAAAAAGGAAAAGAA
ACAAGCCCAAAAGCAGACGAAACTAAGAAAAGAGAAAGAGAAAAAGCCGG
GAAAGATCGCTAAACCTAACACGGCCAATGCGAAGCATACTAGTAGCAAT
TCTAGGGAGATTGCCCAACCATCGTCGAGCAGATACAATGGTGGCAACCA
TAATATCGGCGCAAATAGGTCGAGGTTTAATAATGAAGCGCCCCCTCAAA
CAAGGAAGTTTCAGCCCCCACCAGGTTTTAAAAGAAAATAA
>BUD13 YGL174W SGDID:S000003142,
chrVII:174545 . . . 175345 (SEQ ID NO: 5):
ATGGCATTGCATCAGTATTTATCAGAGACTTATGGGCCCACGAAACCCAA
AAATAAGACGAAAAAGAAGAAGAAAGAGTCAAAATCAGACGCTAACTCAG
ACAAAACTTCTTTGATAGTAAAAGAACGGCTAAGTACACTGCAACAAGAA
CAGGAGAAGTCAGGAGTTGCTTCATTCAGCAAGTTTGACAAACAAAAAAG
CAAGAATATATGGAAGAACCTGGAAACAAACGAGCTTTCCCATGCAATAA
CACATCCTTCCGCATCGTCAATTACTGGCAACGAAAGCAAGAACGATCTA
AAGGAAATCAGGGCTCAAGAGCCACTTGTCACAGTAGCAGACAAATCGAA
AACACGAAAAACCATATACAGAGACGCTCAAGGTCACAAGATTCAGGAAG
ATTCCAAGATAGACGATTCTAGTTTTAGTCGATCTAAATATGAAGATGAG
AAAGCCGCGGAAAGAGAGCAATACCTGAAAAATTTGAATATGGGAGACGT
GCAAAAGCTTGGAATAAATGTAGATGCACATGATAAGAAGAAAAATCAAA
CTGCCTCGAGTCTGACGATAGAAGACCCTGCAATAACATTTACACATGAC
AAAGAAAGAACTGTAAAAACATCTTTACTGGGCCGCAAGCTTTATGATAA
GCCASCACCTGAGAACAGGTTTGCCATTATGCCTGGGTCAAGATSGGACG
GTGTCCACAGATCAAATGGCTTTGAAGAAAAATGGTTTGCTAAGCAAAAT
GAGATCAATGAGAAGAAAGTGCAAAGCTACACCCTACAGGAGGATTATTG
A

The YFR001W gene encodes the 60S ribosomal subunit assembly/export protein LOC1 (UniProtKB—P43586), shown below as SEQ ID NO: 2:

MAPKKPSKRQ NLRREVAPEV FQDSQARNQL ANVPHLTEKS
AQRKPSKTKV KKEQSLARLY GAKKDKKGKY SEKDLNIPTL
NRAIVPGVKI RRGKKGKKFI ADNDTLTLNR LITTIGDKYD
DIAESKLEKA RRLEEIRELK RKEIERKEAL KQDKLEEKKD
EIKKKSSVAR TIRRKNKRDM LKSEAKASES KTEGRKVKKV
SFAQ

The YER029C gene encodes small nuclear ribonucleoprotein-associated protein B (SMB1; UniProtKB—P40018), shown below as SEQ ID NO: 4:

MSKIQVAHSS RLANLIDYKL RVLTQDGRVY IGQLMAFDKH
MNLVLNECIE ERVPKTQLDK LRPRKDSKDG TTLNIKVEKR
VLGLTILRGE QILSTVVEDK PLLSKKERLV RDKKEKKQAQ
KQTKLRKEKE KKPGKIAKPN TANAKHTSSN SREIAQPSSS
RYNGGNDNIG ANRSRFNNEA PPQTRKFQPP PGFKRK

The YGL174W gene encodes pre-mRNA-splicing factor CWC26 (BUD13; UniProtKB—P46947), shown below as SEQ ID NO: 6:

MALHQYLSET YGPTKPKNKT KKKKKESKSD ANSDKTSLIV
KERLSTLQQE QEKSGVASFS KFDKQKSKNI WKNLETNELS
HAITHPSASS ITGNESKNDL KEIRAQEPLV TVADKSKTRK
TIYRDAQGHK IQEDSKIDDS SFSRSKYEDE KAAEREQYLK
NLNMGDVQKL GINVDARDKK KNQTASSLTI EDPAITFTHD
KERTVKTSLL GRKLYDKPAP ENRFAIMPGS RWDGVHRSNG
FEEKWFAKQN EINEKKVQSY TLQEDY

The insertion of the expression cassettes at the jen1D locus was confirmed by colony PCR. The modified yeast strains were grown in non-selective media to remove the plasmid conferring kanamycin resistance used to select transformants, resulting in modified yeast that required no growth supplements compared to the parental yeast. The three strains selected for further study are summarized in Table 4.

TABLE 4
Summary of endogenous protein over-expression strains

| Strain | Gene | Protein |
|---|---|---|
| FG-LOC1^{over} | YFR001W | LOC1 |
| FG-SMB1^{over} | YMR193W | MRPL24 |
| FG-BUD13^{over} | YGL174W | BUD13 |

### Example 3: Production of Lysine by Strains Over-Expressing Lysine-Rich Proteins

Yeast strains overexpressing of LOC1, SMB1 or BUD13 were tested for their ability to produce lysine compared to benchmark yeast; which is wild-type for the LOC1, SMB1 or BUD13 genes; after 24-48 hrs growth in minimum media.

The total protein produced by FG-LOC1^{over}, FG-SMB1^{over}, and FG-BUD13^{over} and parental FG strains were hydrolyzed using acid hydrolysis (6N HCl) at 110° C. for 24 hr (see, e.g., Otter, D. et al. (2012) British Journal of Nutrition 108:S230-S237) and proteogenic lysine content following derivatization using o-plithalaldehyde. Derivatized L-Lysine was detected by HPLC (Agilent Technologies 1260) using an Eclipse Plus C18 column (4.6×150 mm, 3.5-micron) at 40° C. in a gradient of phosphate buffer, pH 7.8 and acetonitrile:methanol:water (45:45:10). Calibration standards used for quantification included known amounts L-Lysine or an amino acid standard mixture (Agilent Technologies) including L-Lysine. Total lysine increase is reported in Table 5 with reference to FG strains.

TABLE 5
Lysine produced by modified and parental yeast

| Strain | K/AA | Fold increase in K |
|---|---|---|
| FG | na | 1.00 |
| FG-LOC1^{over} | 0.2 | 1.58 |
| FG-SMB1^{over} | 0.14 | 1.53 |
| FG-BUD13^{over} | 0.15 | 1.61 |

Yeast-harboring over-expressing of LOC1, BUD13 or SMB1 produced up to 1.6-fold more proteogenic lysine compared to the unmodified reference strain.

### Example 4: Bioavailable Lysine Content of Fermentation Co-Products Using Modified Yeast

The total bioavailable lysine content of fermentation co-products was tested for FG-LOC1^{over} compared to the benchmark strain, FG. Liquefact (corn mash slurry) was prepared by adding 600 ppm of urea, 0.124 SAPU/g ds acid fungal protease, 0.33 GAU/g ds variant Trichoderma reesei glucoamylase and 1.46 SSCU/g ds Aspergillus kawachii α-amylase, adjusted to a pH of 4.8 with sulfuric acid. 100 g of prepared corn liquefact was subjected to fermentation with either FG-LOC1^{over} or the benchmark FG strain at 32° C. with shaking at 200 rpm. After 67 hours, the fermentation broth from duplicate fermentation flasks was collected in an 800-mL beaker and placed into a shaking water bath at 95° C. to evaporate off the ethanol. The fermentation broth was allowed to incubate for approximately 3-5 hours, or until no significant ethanol was detected by HPLC.

The resulting material (i.e., whole stillage) was spun down at 6,000 rpm for 10 min. The supernatant (i.e., thin stillage) and precipitate (i.e., wet cake) were both collected. Wet cake was dried at 37° C. until reaching a dry solids content of about 33-35%. Thin stillage was weighed into 600 mL beakers and put in a shaking water bath at 97° C. to concentrate the contents by about 5-fold (by weight) to create syrup. Water may be added back to beakers to assure that samples were concentrated to the proper, equal degree. To make fermentation co-products similar to DDGS samples, wet cake and the corresponding syrup were combined at a 2-to-1 mass ratio (as-is weights) and mixed well. DDGS was spread onto a metal tray and dried in a 99° C. oven for about 3 hours, with occasional mixing to >90% dry solids content.

To test for bioavailable amino acids, samples of DDGS were incubated with pepsin and pancreatin, based on a previously reported method (Qiao Y (2001) Routine techniques for monitoring the nutritional value of animal meals, Doctoral thesis at North Carolina State University). Briefly, 0.33 g of DDGS was added to a 20 mL scintillation vial along with 3.33 mL of 0.05 M citrate buffer (pH 2) and approximately 0.012 g pepsin (from porcine gastric mucosa) at ≥400 units/mg protein. The mixture was allowed to incubate at 38° C. for about 24 hours with shaking at 200 rpm. After this time, 5 mL of phosphate buffer (0.2 M, pH 11.5, with 0.025% w/w sodium azide) and approximately 0.023 g pancreatin (from porcine pancreas, 4× UXP specifications) was added to each vial. The vials were placed back into the 38° C. incubator shaking with at 200 rpm for around 66 hours. After this time, samples were taken from each vial, spun down through a 0.2 μM filter and analyzed by HPLC for free amino acids.

Bioavailable lysine content in the fermentation co-product produced by fermentation with the FG-LOC1^{over} strain was 1.08-fold greater (i.e., 8% greater) compared to the lysine content in the fermentation co-product produced by fermentation with the parental FG strain.

What is claimed is:

1. A microorganism for use in preparing a food or feed composition, comprising a genetic modification that increases the expression of an endogenous gene encoding a protein having an elevated ratio of a preselected amino acid relative to the total amino acid content of the protein, wherein the preselected amino acid confers a nutritional benefit to the food or feed composition compared an otherwise identical food or feed composition comprising an otherwise identical microorganism, or product derived therefrom, lacking the genetic modification.

2. The microorganism of claim 1, wherein the endogenous gene is naturally present n the microorganism prior to introducing the genetic modification.

3. The microorganism of claim 1 or 2, wherein the genetic modification is the introduction of an expression cassette comprising an additional copy of the endogenous gene.

4. The microorganism of claim 1 or 2, wherein the genetic modification is the introduction of a stronger promoter operably-linked to the endogenous gene.

5. The microorganism of claim 1 or 2, wherein the genetic modification is the deletion of a naturally-present negative regulator of expression of the endogenous gene, or wherein the genetic modification increases the expression of a naturally-present positive regulator of expression of the endogenous gene.

6. The microorganism of any of claims 1-5, wherein the elevated ratio of the preselected amino acid relative to the total amino acid content of the protein is at least 1.2 compared to the ratio of the preselected amino acid relative to the total amino acid content of all proteins produced by the microorganism.

7. The microorganism of any of claims 1-6, wherein the organism is an ethanolagen.

8. The microorganism of any of claims 1-7, wherein the organism is a Saccharomyces sp.

9. The microorganism of any of claims 1-8, wherein the microorganism does not comprise an exogenous gene encoding a protein having an elevated ratio of a preselected amino acid relative to the total amino acid content of the protein that is introduced for the purpose of conferring a nutritional benefit to the food or feed composition.

10. The microorganism of any of claims 1-9, wherein the cells further comprise an exogenous gene encoding a carbohydrate processing enzyme, one or more genes of the phosphoketolase pathway, an alteration in the glycerol pathway and/or the acetyl-CoA pathway, or an alternative pathway for making ethanol.

11. A method for increasing the nutritional value of a microorganism, or product derived therefrom, in a food or feed composition, comprising introducing into the microorganism a genetic modification that increases the expression of an endogenous gene encoding a protein having an elevated ratio of a preselected amino acid relative to the total amino acid content of the protein, wherein the preselected amino acid confers a nutritional benefit to the food or feed composition compared an otherwise identical food or feed composition comprising an otherwise identical microorganism, or product derived therefrom, lacking the genetic modification.

12. The method of claim 11, wherein the endogenous gene is naturally present in the microorganism prior to introducing the genetic modification.

13. The method of claim 11 or 12, wherein the genetic modification is the introduction of an expression cassette comprising an additional copy of the endogenous gene.

14. The method of claim 11 or 12, wherein the genetic modification is the introduction of a stronger promoter operably-linked to the endogenous gene.

15. The method of claim 11 or 12, wherein the genetic modification is the deletion of a naturally-present negative regulator of expression of the endogenous gene, or wherein the genetic modification increases the expression of a naturally-present positive regulator of expression of the endogenous gene.

16. The method of any of claims 11-15, wherein the elevated ratio of the preselected amino acid relative to the total amino acid content of the protein is at least 1.2 compared to the ratio of the preselected amino acid relative to the total amino acid content of all proteins produced by the microorganism.

17. The method of any of claims 11-16, wherein the microorganism is an ethanolagen.

18. The method of any of claims 11-17, wherein the organism is a Saccharomyces sp.

19. The method of any of claims 11-18, wherein the microorganism does not comprise an exogenous gene encoding a protein having an elevated ratio of a preselected amino acid relative to the total amino acid content of the protein that is introduced for the purpose of, conferring a nutritional benefit to the food or feed composition.

20. The method of any of claims 11-19, wherein the cells further comprise an exogenous gene encoding a carbohydrate processing enzyme, one or more genes of the phosphoketolase pathway, an alteration in the glycerol pathway and/or the acetyl-CoA pathway, or an alternative pathway for making ethanol.
